(19) United States
(12) Patent Application Publication
(10) Pub. No.: US 2026/0209679 A1
(43) Pub. Date: Jul. 23, 2026

(54) CHLORINE DIOXIDE CONCENTRATION CONTROL SYSTEM

(71) Applicant: TAIKO PHARMACEUTICAL CO., LTD., Osaka (JP)

(72) Inventors: Koushirou SOGAWA, Osaka (JP); Kouichi TAURA, Osaka (JP); Kazuhiko TAGUCHI, Osaka (JP); Kenkichi AOKI, Osaka (JP)

(21) Appl. No.: 19/140,175

(22) PCT Filed: Dec. 5, 2023
(86) PCT No.: PCT/JP2023/043375

§ 371 Date: Jun. 17, 2025

(30) Foreign Application Priority Data
Dec. 19, 2022 (JP) 2022-201886

(51) Int. Cl. C12M 1/34; C12M 1/00; C12M 1/12; C12M 1/36; C25B 1/26

(52) U.S. Cl. CPC C12M 41/34; C12M 29/06; C12M 37/06; C12M 41/14; C12M 41/48; C25B 1/26

(57) ABSTRACT

The object of the present invention is to realize sanitary control at cell culture processing facilities with chlorine dioxide gas at a low concentration. An automatic chlorine dioxide concentration control system in a cell culture device, comprising a chlorine dioxide feeding device and a chlorine dioxide concentration measuring device is provided.

### TECHNICAL FIELD

The present invention relates to a chlorine dioxide concentration control system.

### BACKGROUND ART

In recent years, the importance of cell culture processing facilities is growing with the fast expansion of the biopharmaceutical market. Strict sanitary control is required in order to manufacture homogeneous cell products at cell culture processing facilities. However, it is difficult to fully prevent contamination of microorganisms into cultured cells.

Sogawa, et al., 2020 (Non-Patent Literature 1) and Okawa, et al., 2022 (Non-Patent Literature 2) propose that by employing chlorine dioxide gas at a low concentration, contamination by microorganisms is prevented without inflicting adverse effect to cell growth.

### CITATION LIST

### Non-patent Literatures

[Non-Patent Literature 1] Regenerative Therapy 14(2020 ) 184-190
[Non-Patent Literature 2] Regenerative Therapy 21(2022 ) 250-257

### SUMMARY OF THE INVENTION

### Problems to be Solved by the Invention

Chlorine dioxide gas is known to be a safe gas for the living animal body at low concentrations (such as 0.1 ppm or lower), while having inactivation effect against microorganisms such as bacteria, fungus, and virus, as well as deodorization effect, and the like, even at such low concentrations. However, when the chlorine dioxide gas concentration becomes high to a certain level or higher, adverse effects may develop for workers, and when the chlorine dioxide gas concentration becomes low to a certain level or lower, the desired inactivation effect against microorganisms may not be obtained. Accordingly, in order to realize sanitary control at cell culture processing facilities with chlorine dioxide gas at a low concentration, a chlorine dioxide concentration control system is essential.

### Means for Solving the Problems

In order to solve the above problems, the present inventors performed repeated research to succeed in developing an automatic chlorine dioxide concentration control system.

In other words, in one embodiment, the present invention relates to an automatic chlorine dioxide concentration control system in a cell culture device, wherein
said system comprises:
(i) a chlorine dioxide feeding device comprising a first automatic controlling valve, and
(ii) a chlorine dioxide concentration measuring device comprising a chlorine dioxide concentration measuring sensor;
   said chlorine dioxide feeding device and said chlorine dioxide concentration measuring device are each connected to be in fluid connection with said cell culture device; and
   when the chlorine dioxide concentration in said cell culture device measured by said chlorine dioxide concentration measuring device falls under predetermined first value, said first automatic controlling valve is opened to feed chlorine dioxide gas from said chlorine dioxide feeding device into said cell culture device, and when the chlorine dioxide concentration in said cell culture device measured by said chlorine dioxide concentration measuring device exceeds a predetermined second value, said first automatic controlling valve is closed to stop the feeding of chlorine dioxide gas from said chlorine dioxide feeding device into said cell culture device, thereby maintaining the chlorine dioxide concentration in said cell culture device to generally within the range of said first value and said second value.

One embodiment of the present invention is characterized in that said chlorine dioxide feeding device is a pressure-resistant container comprising a nozzle or piping that cooperates with the first automatic controlling valve, and said pressure-resistant container is filled with chlorine dioxide along with a carrier gas.

One embodiment of the present invention is characterized in that said carrier gas is liquified carbon dioxide, and chlorine dioxide is dissolved in said liquified carbon dioxide.

One embodiment of the present invention is characterized in that the concentration of said chlorine dioxide in said liquified carbon dioxide is 1×10⁻²−1×10⁵ ppm.

One embodiment of the present invention is characterized in that said pressure-resistant container is a gas cylinder or a gas tank.

One embodiment of the present invention is characterized in that said chlorine dioxide feeding device is a chlorine dioxide feeding device that produces chlorine dioxide gas by electrolyzing an electrolytic solution comprising chlorites.

One embodiment of the present invention is characterized in that said chlorite is selected from the group consisting of sodium chlorite, potassium chlorite, lithium chlorite, calcium chlorite, magnesium chlorite, and barium chlorite.

One embodiment of the present invention is characterized in that said electrolytic solution comprises 0.1% by weight-30% by weight of chlorites.

One embodiment of the present invention is characterized in that said chlorine dioxide concentration measuring device comprises two or more chlorine dioxide concentration measuring sensors.

One embodiment of the present invention is characterized in that said two or more chlorine dioxide concentration measuring sensors are connected in parallel to said cell culture device, and when one chlorine dioxide concentration measuring sensor is used for measuring the chlorine dioxide concentration in said cell culture device, the other chlorine dioxide concentration measuring sensor is ventilated.

One embodiment of the present invention is characterized in that the ventilation of the said other chlorine dioxide concentration measuring sensor is performed by zero gas (gas for performing zero calibration).

One embodiment of the present invention is characterized in that the switching of said two or more chlorine dioxide concentration measuring sensors is performed at regular intervals by one or more automatic controlling valves that are different from said first automatic controlling valve.

One embodiment of the present invention is characterized in that said first automatic controlling valve and said chlorine dioxide concentration measuring device are in cooperation with each other by wire or wirelessly.

One embodiment of the present invention is characterized in that said automatic controlling valve is an electromagnetic valve or an electric valve.

One embodiment of the present invention is characterized in that said cell culture device is a CO₂ incubator.

An invention of any combination of one or more characteristics of the present invention listed above is also encompassed by the scope of the present invention.

### Effects of the Invention

According to the system of the present invention, the chlorine dioxide concentration in the cell culture device can be controlled to within a given range, and contamination of microorganisms in cells cultured or processed inside the cell culture device can be prevented. Moreover, according to the System of the present invention, since the chlorine dioxide concentration in the cell culture device can be automatically controlled, a cultured cell of stable quality compared to when manually controlling the chlorine dioxide concentration can be obtained.

### BRIEF DESCRIPTION OF THE DRAWINGS

FIG. 1 shows the most basic configuration of the system of the present invention (Configuration Example 1).

FIG. 2 shows a flow chart related to the basic actions of the system of the present invention.

FIG. 3 shows Configuration Example 2 of the system of the present invention (a pressure-resistant container-type chlorine dioxide feeding device).

FIG. 4 shows Configuration Example 3 the system of the present invention (an electrolysis-type chlorine dioxide feeding device).

FIG. 5 shows Configuration Example 4 the system of the present invention (a pressure-resistant container-type chlorine dioxide feeding device+two chlorine dioxide concentration measuring sensors).

FIG. 6 shows Configuration Example 5 the system of the present invention (an electrolysis-type chlorine dioxide feeding device+two chlorine dioxide concentration measuring sensors).

FIG. 7 shows the measurement data of the chlorine dioxide concentration in the cell culture device employing the system of the present invention.

### DESCRIPTION OF EMBODIMENTS

The embodiments for carrying out the present invention will now be described with the Configuration Examples shown in each drawing.

### Configuration Example 1

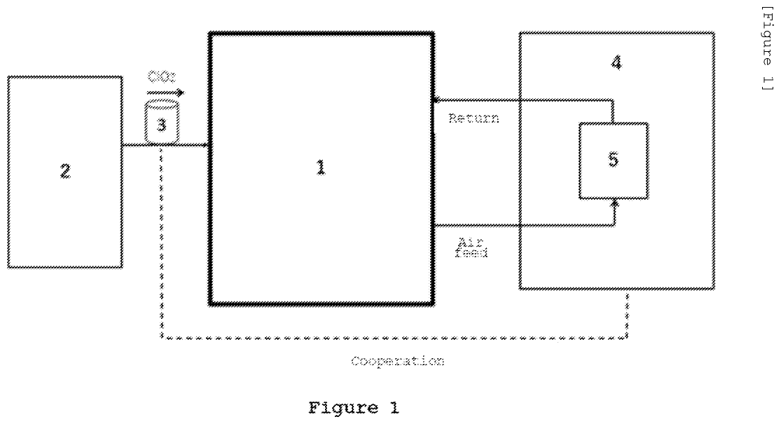

FIG. 1 shows an automatic chlorine dioxide concentration control system in a cell culture device according to one embodiment of the present invention. Said system comprises: (i) a chlorine dioxide feeding device 2 comprising an automatic controlling valve 3, and (ii) a chlorine dioxide concentration measuring device 4 comprising a chlorine dioxide concentration measuring sensor 5; wherein the chlorine dioxide feeding device 2 and the chlorine dioxide concentration measuring device 4 are each connected to be in fluid connection with said cell culture device 1.

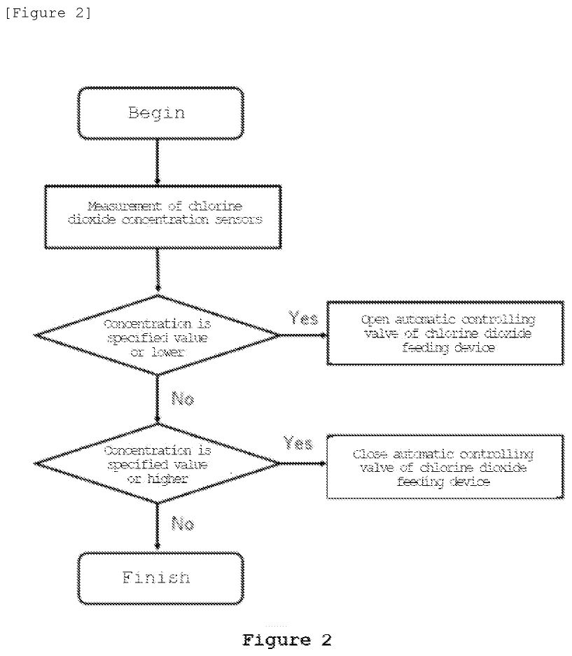

The chlorine dioxide concentration measuring device 4 (and/or the chlorine dioxide concentration measuring sensor 5) and the automatic controlling valve 3 may be in cooperation with each other by wire or wirelessly, and as shown in the flow chart of FIG. 2, may be set so that when the chlorine dioxide concentration measured by the chlorine dioxide concentration measuring device 4 becomes a specified value or lower, the automatic controlling valve 3 is opened, and when the chlorine dioxide concentration measured by the chlorine dioxide concentration measuring device 4 becomes a specified value or higher, the automatic controlling valve 3 is closed. Note that the cooperation of the chlorine dioxide concentration measuring device 4 (and/or the chlorine dioxide concentration measuring sensor 5) and the automatic controlling valve 3 may be in cooperation mode where the automatic controlling valve 3 directly receives the information sent from the chlorine dioxide concentration measuring device 4 or the chlorine dioxide concentration measuring sensor 5, or may be in cooperation mode where information is indirectly transmitted via one or more computers.

In the system shown in FIG. 1, when the chlorine dioxide concentration in the cell culture device 1 measured by the chlorine dioxide concentration measuring device 4 falls under the predetermined first value, the automatic controlling valve 3 is opened to feed chlorine dioxide gas from the chlorine dioxide feeding device 2 into the cell culture device 1, and when the chlorine dioxide concentration in the cell culture device 1 measured by the chlorine dioxide concentration measuring device 4 exceeds the predetermined second value, the automatic controlling valve 3 is closed to stop the feeding of chlorine dioxide gas from the chlorine dioxide feeding device 2 into the cell culture device 1, thereby maintaining the chlorine dioxide concentration in the cell culture device 1 to generally within the range of said first value and said second value.

Note that in the present disclosure, when stated that “the chlorine dioxide concentration inside the cell culture device is maintained to generally within the range of the first value and the second value,” it may mean that, for example, for a duration of 90% or longer (or 91% or longer, 92% or longer, 93% or longer, 94% or longer, 95% or longer, 96% or longer, 97% or longer, 98% or longer, 99% or longer) of the operation duration of the system of the present invention, the chlorine dioxide concentration inside the cell culture device is maintained to within the range of the first value and the second value.

The chlorine dioxide feeding device in the present disclosure may be, for example, a device that feeds the chlorine dioxide which is stored in the interior of the device to the exterior, or may be a device that produces chlorine dioxide in the interior of the device and feeds it to the exterior.

The cell culture device to which the system of the present invention may be applied may be, for example, an equipment that is used for culturing and processing of cells at cell culture processing facilities etc., and preferably may be an CO₂ incubator.

The automatic controlling valve used in the system of the present invention may be, for example, an automatic valve that is driven by electricity when a given information is input, preferably may be an electromagnetic valve that is driven by an actuator which uses an electromagnet, and may be an electric valve that is driven by a motor. The type of electromagnetic valve or electric valve that may be used for the system of the present invention is not limited, and a commercially available electromagnetic valve or electric valve may be used. Non-limiting specific examples that can be employed include, for example, an electromagnetic valve AVH-4345 (AC-100 V) or AVH-4342 (DC-24 V) from ADVANCE ELECTRIC CO., INC.

### Configuration Example 2

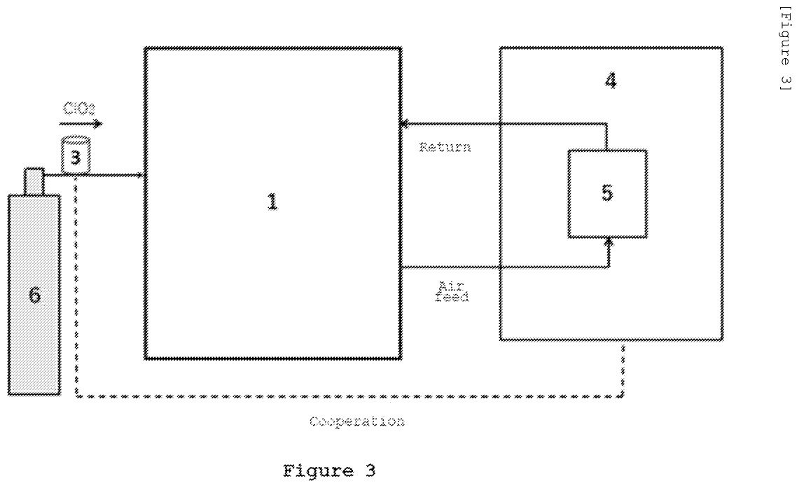

FIG. 3 shows, as the chlorine dioxide feeding device of Configuration Example 1, an example employing a pressure-resistant container 6 filled with chlorine dioxide. The chlorine dioxide filled in the pressure-resistant container 6 is fed to the cell culture device 1 through a nozzle or piping. The feed/stop of the gas filled in the pressure-resistant container 6 is controlled by the automatic controlling valve 3 that directly or indirectly cooperates with the nozzle or piping.

An example of a device that feeds the chlorine dioxide which is stored in the interior of the device to the exterior that may be employed in the present invention can include a pressure-resistant container filled with chlorine dioxide along with a carrier gas. As carrier gas, it is preferred to employ a gas that does not chemically react with chlorine dioxide, and for example, an inert gas such as carbon dioxide, nitrogen gas, argon, and helium, air, and the like may be employed. Most preferably, as chlorine dioxide feeding device, a pressure-resistant container filled with chlorine dioxide gas in a state dissolved in liquified carbon dioxide may be used. By allowing chlorine dioxide to exist in a state dissolved in liquified carbon dioxide inside the pressure-resistant container, it is possible to prevent degradation of chlorine dioxide, and to store chlorine dioxide in a stable state. Moreover, by allowing chlorine dioxide to exist in a state dissolved in liquified carbon dioxide inside the pressure-resistant container, it is also possible to prevent the pressure-resistant container from being corroded by chlorine dioxide.

If the pressure-resistant container filled with chlorine dioxide gas in a state dissolved in liquified carbon dioxide is used as the chlorine dioxide feeding device, since when the mixture of chlorine dioxide gas and liquified carbon dioxide is released from the device, the liquified carbon dioxide immediately vaporizes and expands, the chlorine dioxide gas can be very efficiency diffused. Moreover, since a constant concentration of chlorine dioxide gas is constantly present inside the pressure-resistant container, if the chlorine dioxide gas concentration inside the device is temporarily reduced by opening and closing the cell culture device etc., a required amount of gas can be rapidly fed.

The method for manufacturing a pressure-resistant container filled with chlorine dioxide gas in a state dissolved in liquified carbon dioxide is not limited, and for example, may be a method comprising a step of filling chlorine dioxide into a pressure-resistant container and a step of filling liquified carbon dioxide into a pressure-resistant container filled with chlorine dioxide while dissolving the chlorine dioxide in the liquified carbon dioxide. Another exemplary method for manufacturing may be a method comprising a step of filling chlorine dioxide along with carbon dioxide into a pressure-resistant container.

The pressure-resistant container may be for example a commercially available gas cylinder or gas tank, and a siphon-type gas cylinder that takes out the liquified carbon dioxide having chlorine dioxide dissolved therein in liquid phase state may be employed, or a common gas cylinder that takes out the liquified carbon dioxide having chlorine dioxide dissolved therein in gas phase state may be employed. If a common gas cylinder is used in an inverted state, it is also possible to take out the liquified carbon dioxide having chlorine dioxide dissolved therein in liquid phase state.

The concentration of chlorine dioxide filled into the pressure-resistant container is not limited, and for example, the concentration of chlorine dioxide in liquified carbon dioxide may be set at 1×10⁻²−1×10⁵ ppm (more preferably 1×10⁻¹−1×10⁴ ppm, further preferably 1×10⁻¹−5×10³ ppm).

### Configuration Example 3

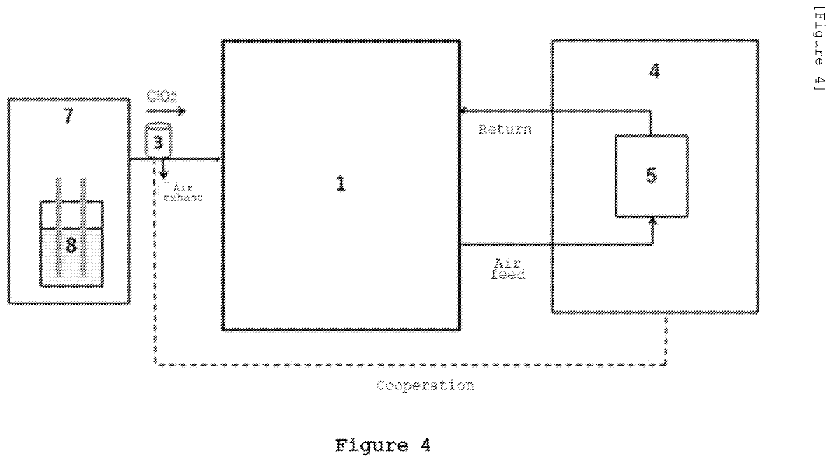

FIG. 4 shows, as the chlorine dioxide feeding device of Configuration Example 1, an example employing an electrolysis-type chlorine dioxide generator 7. The chlorine dioxide generator 7 may comprise an electrolysis tank 8 for electrolyzing an electrolytic solution comprising chlorites, and the chlorine dioxide gas produced by the chlorine dioxide generator 7 is fed to the cell culture device 1 through a piping. The feed/stop of the gas produced from the chlorine dioxide generator 7 is controlled by the automatic controlling valve 3 that directly or indirectly cooperates with the piping. Note that since the electrolysis tank 8 continuously produces chlorine dioxide gas, when stopping the feed of gas to the cell culture device 1, the gas produced may be exhausted to the exterior of the device.

The device that produces chlorine dioxide in the interior of the device and feeds it to the exterior that may be employed in the present invention may be for example an electrolysis-type chlorine dioxide generator. The configuration of the electrolysis-type chlorine dioxide generator is not limited, and may be for example a chlorine dioxide generator that performs electrolysis by feeding direct electric current to the electrolytic solution inside a non-membrane electrolytic tank comprising a cathode and an anode.

The electrolytic solution that may be used in the electrolysis-type chlorine dioxide generator may be an electrolytic solution comprising 0.1% by weight-30% by weight of chlorites. When the proportion of chlorites in the electrolytic solution is less than 0.1% by weight, the chlorite necessary for electrolysis will fall short and there will be a possibility that a sufficient amount of chlorine dioxide will not be produced. When the proportion of chlorites in the electrolytic solution exceeds 30% by weight, there will be a possibility that the chlorites saturate and crystals precipitate, and the efficiency of electrolysis will be reduced. A preferred proportion of chlorites in the electrolytic solution is 1% by weight-10% by weight, and a further preferred proportion is 1% by weight-3% by weight.

The types of chlorites that may be comprised in the electrolytic solution is not limited, and may be for example a chlorite selected from the group consisting of sodium chlorite, potassium chlorite, lithium chlorite, calcium chlorite, magnesium chlorite, and barium chlorite.

The electrolytic solution that may be used in the electrolysis-type chlorine dioxide generator may further comprise alkali chlorides. Chlorine gas is produced when alkali chlorides are electrolyzed, and the chlorine gas produced immediately reacts with chlorites to become chlorine dioxide. The proportion of the alkali chlorides in the electrolytic solution is preferably 1% by weight or more, and further preferably 2% by weight or more (less than the degree of solubility). When the proportion of the alkali chlorides in the electrolytic solution is less than 1% by weight, the chlorine gas may not be sufficiently produced, and the production efficiency of chlorine dioxide may be reduced. When the alkali chloride concentration in the electrolytic solution is increased, chlorine dioxide can be efficiently produced, but the alkali chlorides precipitate in the electrolytic solution and the efficiency of electrolysis will be reduced when it becomes higher than the degree of solubility. For this reason, it is preferred that the proportion of the alkali chlorides in the electrolytic solution is approximately 20% by weight or less, although it is not unconditional since it varies depending on the type of alkali chloride or the temperature of the electrolytic solution.

The type of alkali chloride that may be comprised in the electrolytic solution is not limited, and may be, for example, potassium chloride, sodium chloride, lithium chloride, or calcium chloride.

The chlorine dioxide produced inside the reaction tank by electrolysis is for example collected by bubbling the electrolytic solution with a carrier gas that that does not chemically react with chlorine dioxide, and can be utilized for the system of the present invention. As carrier gas, for example, an inert gas such as carbon dioxide, nitrogen gas, argon, and helium, air, and the like may be employed.

### Configuration Examples 4 and 5

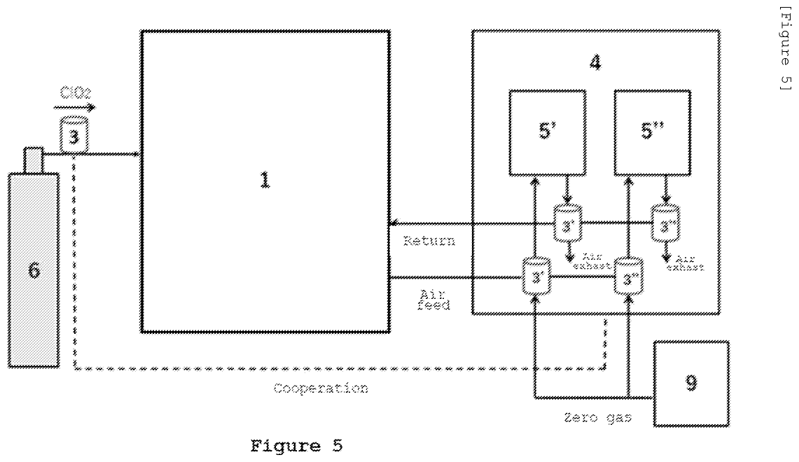

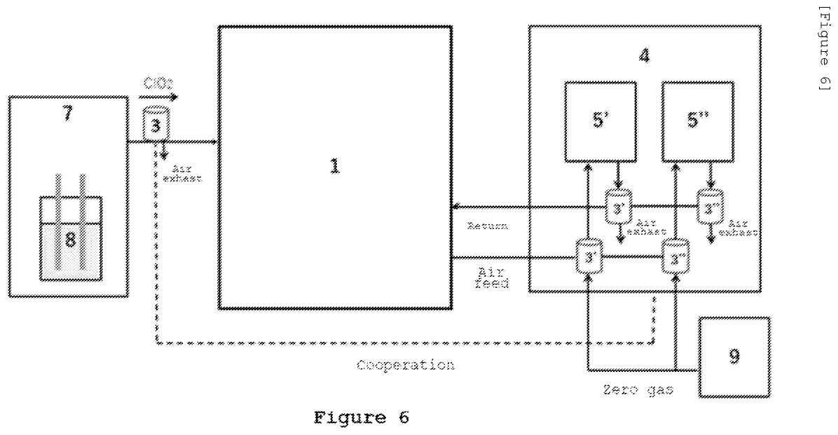

FIG. 5 and FIG. 6 shows examples of using the chlorine dioxide concentration measuring device in Configuration Examples 3 and 4 as the chlorine dioxide concentration measuring device comprising two chlorine dioxide concentration measuring sensors (5′, 5″).

The chlorine dioxide concentration measuring device in the system of the present invention may be implemented with only one chlorine dioxide concentration measuring sensor, but it may also comprise two or more chlorine dioxide concentration measuring sensors. Since the inside of the chlorine dioxide concentration measuring sensor is inclined to be of generally high humidity, sensor action can be stabilized by having a system that alternately uses the two or more chlorine dioxide concentration measuring sensors. Note that the chlorine dioxide concentration measuring sensor that may be used in the present invention is not limited as long as it is commercially available, and for example, GD-70D from RIKEN KEIKI CO., LTD. can be used.

As illustrated in FIG. 5 and FIG. 6, when comprising a chlorine dioxide concentration measuring device comprising two or more chlorine dioxide concentration measuring sensors (5′, 5″), by using one chlorine dioxide concentration measuring sensor 5′ for measurement of the chlorine dioxide concentration in the cell culture device 1, while running zero gas through the other chlorine dioxide concentration measuring sensor 5″, sensor 5″ can be subjected to ventilation and zero calibration. It is preferred that the switching of the sensors used for measurement of chlorine dioxide concentration is performed by the automatic controlling valves 3′, 3″ at regular intervals. In this way, by employing the alternately used two or more chlorine dioxide concentration measuring sensors, measurement of chlorine dioxide concentration can be further stabilized.

The terms used herein are employed for describing particular embodiments, and do not intend to limit the invention.

Moreover, the term “comprising” as used herein, unless the content clearly indicates to be recognized otherwise, intends the presence of the described items (such as components, steps, elements, or numbers), and does not exclude the presence of other items (such as components, steps, elements, and numbers).

Unless otherwise defined, all terms used herein (including technical and scientific terms) have the same meanings as those broadly recognized by those skilled in the art of the technology to which the present invention belongs. The terms used herein, unless explicitly defined otherwise, should be construed as having meanings consistent with the meanings herein and in related technical fields, and shall not be construed as having idealized or excessively formal meanings.

The embodiments of the present invention may be described with reference to schematic diagrams. In such a case, they may be exaggerated in presentation in order to allow clear description.

In the present specification, for example when the expression “1-10% by weight” is used, those skilled in the art will recognize that the aforementioned expression individually and specifically refers to 1, 2, 3, 4, 5, 6, 7, 8, 9, or 10% by weight.

In the present specification, any and all numeric values employed to indicate component contents or numeric value ranges are, unless explicitly indicated, to be construed as encompassing the meaning of the term “about.” For example, “10-folds, ” unless explicitly indicated, is recognized to mean “about 10-folds.”

All of the disclosures of the literatures cited herein should be deemed as cited herein, and those skilled in the art will cite and recognize the related disclosed contents in these prior art literatures as a part of the present specification according to the context herein without departing from the spirit and scope of the present invention.

### EXAMPLES

### Example 1

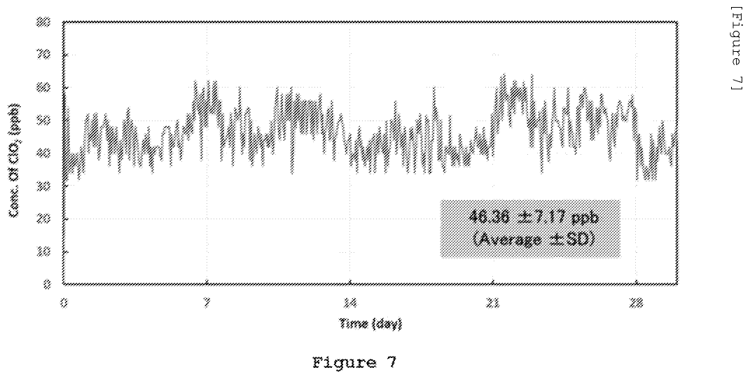

The data of actually performing the chlorine dioxide concentration control test of the cell culture device using the chlorine dioxide concentration control system of the present invention is shown in FIG. 7. The test was performed with the automatic chlorine dioxide concentration control system of Configuration Example 4 (FIG. 5) which is one embodiment of the present invention. The system was set so that when the chlorine dioxide gas concentration inside the cell culture device falls under 40 ppb, the automatic controlling valve is opened to feed chlorine dioxide gas from the chlorine dioxide feeding device into the cell culture device, and when the chlorine dioxide concentration inside the cell culture device exceeds 60 ppb, the automatic controlling valve is closed to stop the feeding of chlorine dioxide gas from the chlorine dioxide feeding device into the cell culture device. Note that GD-70D from RIKEN KEIKI CO., LTD. was employed as the chlorine dioxide concentration measuring sensor, and electromagnetic valves (AVH-4345, AVH-4342) from ADVANCE ELECTRIC CO., INC. were employed as the automatic controlling valves.

As shown in FIG. 7, by employing the system of the present invention, the chlorine dioxide concentration inside the cell culture device could be maintained to a desired range for a long period of time.

### DESCRIPTION OF SYMBOLS

1 Cell culture device
2 Chlorine dioxide feeding device
3, 3′, 3″ Automatic controlling valve
4 Chlorine dioxide concentration measuring device
5, 5′, 5″ Chlorine dioxide concentration measuring sensor
6 Pressure-resistant container filled with chlorine dioxide
7 Chlorine dioxide feeding device comprising electrolysis tank
8 Electrolysis tank
9 Zero gas filter

1. An automatic chlorine dioxide concentration control system in a cell culture device, wherein
said system comprises:
(i) a chlorine dioxide feeding device comprising a first automatic controlling valve, and
(ii) a chlorine dioxide concentration measuring device comprising a chlorine dioxide concentration measuring sensor;
said chlorine dioxide feeding device and said chlorine dioxide concentration measuring device are each connected to be in fluid connection with said cell culture device; and
when the chlorine dioxide concentration in said cell culture device measured by said chlorine dioxide concentration measuring device falls under predetermined first value, said first automatic controlling valve is opened to feed chlorine dioxide gas from said chlorine dioxide feeding device into said cell culture device, and when the chlorine dioxide concentration in said cell culture device measured by said chlorine dioxide concentration measuring device exceeds a predetermined second value, said first automatic controlling valve is closed to stop the feeding of chlorine dioxide gas from said chlorine dioxide feeding device into said cell culture device, thereby maintaining the chlorine dioxide concentration in said cell culture device to generally within the range of said first value and said second value.

2. The system according to claim 1, wherein said chlorine dioxide feeding device is a pressure-resistant container comprising a nozzle or piping that cooperates with the first automatic controlling valve, and said pressure-resistant container is filled with chlorine dioxide along with a carrier gas.

3. The system according to claim 2, wherein said carrier gas is liquified carbon dioxide, and chlorine dioxide is dissolved in said liquified carbon dioxide.

4. The system according to claim 3, wherein the concentration of said chlorine dioxide in said liquified carbon dioxide is 1×10⁻²−1×10⁵ ppm.

5. The system according to claim 2, wherein said pressure-resistant container is a gas cylinder or a gas tank.

6. The system according to claim 1, wherein said chlorine dioxide feeding device is a chlorine dioxide feeding device that produces chlorine dioxide gas by electrolyzing an electrolytic solution comprising chlorites.

7. The system according to claim 6, wherein said chlorite is selected from the group consisting of sodium chlorite, potassium chlorite, lithium chlorite, calcium chlorite, magnesium chlorite, and barium chlorite.

8. The system according to claim 6, wherein said electrolytic solution comprises 0.1% by weight- 30% by weight of chlorites.

9. The system according to claim 1, wherein said chlorine dioxide concentration measuring device comprises two or more chlorine dioxide concentration measuring sensors.

10. The system according to claim 9, wherein
said two or more chlorine dioxide concentration measuring sensors are connected in parallel to said cell culture device, and
when one chlorine dioxide concentration measuring sensor is used for measuring the chlorine dioxide concentration in said cell culture device, the other chlorine dioxide concentration measuring sensor is ventilated.

11. The system according to claim 10, wherein the ventilation of the said other chlorine dioxide concentration measuring sensor is performed by zero gas.

12. The system according to claim 10, wherein the switching of said two or more chlorine dioxide concentration measuring sensors is performed at regular intervals by one or more automatic controlling valves that are different from said first automatic controlling valve.

13. The system according to claim 1, wherein said first automatic controlling valve and said chlorine dioxide concentration measuring device are in cooperation with each other by wire or wirelessly.

14. The system according to claim 1, wherein said automatic controlling valve is an electromagnetic valve or an electric valve.

15. The system according to claim 1, wherein said cell culture device is a CO₂ incubator.
